SEQUENCE LISTING (17 sequences)

SEQ ID NO: 1 - DNA, 684 bp, Arabidopsis thaliana
  CDS (1)..(684)
atgccggtgcacgatgagcaagagcatgaggtgtacggtggagagatcccagaagaggaagaaggtgagatggatacggaagagtacgaagaacacggcggagaagaaggtgccgccgccggagatgaggagcttgaacctggctctagctctagggacttggaggatatgaagaaacgaattaaggagattgaggaagaagctggagctttgcgtgaaatgcaagccaaggctgagaaagatatgggtgctagtcaagacccttcaggtggtgttagtgcggctgagaaggaagaagtggattcgcgttcaatatatgttggcaatgtggactatgcatgtactccagaggaagtccagcagcattttcaatcatgtgggacagtgaatagggttacaattctgacagataagtttggtcaacctaaaggttttgcgtatgtcgaatttgtggaagtagaagctgttcagaattcgcttattttgaacgagtcagaactacatggtcgtcagataaaggtatctgcaaagaggactaatgtacctggaatgaggcaatttcgaggaagagggcgtccctttagaccgatgaggggattcatgccaggagttccattttatcctccatatgcttatgggagagttcccaggttcagacggcctatgcgctacagaccgtactga

SEQ ID NO: 2 - protein, 227 aa, Arabidopsis thaliana
MPVHDEQEHEVYGGEIPEEEEGEMDTEEYEEHGGEEGAAAGDEELEPGSSSRDLEDMKKRIKEIEEEAGALREMQAKAEKDMGASQDPSGGVSAAEKEEVDSRSIYVGNVDYACTPEEVQQHFQSCGTVNRVTILTDKFGQPKGFAYVEFVEVEAVQNSLILNESELHGRQIKVSAKRTNVPGMRQFRGRGRPFRPMRGFMPGVPFYPPYAYGRVPRFRRPMRYRPY

SEQ ID NO: 3 - DNA, 630 bp, Triticum aestivum
  CDS (1)..(630)
atggaggacgaagagcacgaggtgtacggccaggagatccccgtcgacggcgaggacgtcgacatgggcgccgggggcgacgacgccgccaagctgcacgagctcgacgagatgaagcgccgtctcaaggagatggaggaggaggccgccgccctccgcgagatgcaggccaaggtcgccaaggagatgcaaggcggagatcctaatgcgaccacatctgaagcaaaagaagagatggattctcgttctgtttttgttggaaatgttgactatgcatgcactccagaagaagtgcagcagcattttaattcgtgcggaaccgtcaaccgagtgactatcctgaccgacaagtttgggcagcctaaaggttttgcttacgttgaatttgttgaagcagaagctattcaggaagctgtcaagctgagtgagtcagagcttcatggtcggcaaatcaaggtagcgccgaagcgaactaacgttcctgggttgaagcagccccgagggggccgaggattcaacccatatggcggccacccctacatgaggccatatcctccaccgttttacaatccttatggtggttatgggagagctcccaggttccgccgtccgcgcaggcccttctactaa

SEQ ID NO: 4 - protein, 209 aa, Triticum aestivum
MEDEEHEVYGQEIPVDGEDVDMGAGGDDAAKLHELDEMKRRLKEMEEEAAALREMQAKVAKEMQGGDPNATTSEAKEEMDSRSVFVGNVDYACTPEEVQQHFNSCGTVNRVTILTDKFGQPKGFAYVEFVEAEAIQEAVKLSESELHGRQIKVAPKRTNVPGLKQPRGGRGFNPYGGHPYMRPYPPPFYNPYGGYGRAPRFRRPRRPFY

SEQ ID NO: 5 - DNA, 633 bp, Triticum aestivum
  CDS (1)..(633)
atggacgaggaggagcacgaggtgtacggccaggagatccccgtggacggcgacatggaagccgccgacgtcgacatgaccgccgccggagacgacgcagcgaaggagctggacgagatgaagcgaaggctcaaggagatggaggaggaggccgccgccctgcgcgacatgcaggccaaggtcgccaaggagatgcaaggaggagatgccaatgcgtctacagctgaggcaaaggagcaggtggatgctcgctctgtttatgtcggaaatgttgattatgcgtgtacaccagaggaagtgcagcagcatttccaagcttgtggaactgtcaacagagtgacaatcttgaccgacaagtttggacagccaaaaggatttgcttacgtggaatttctagaacaagaagccgttcaggaggctcttaatctgaatgaatctgaattgcatggtcgtcagatcaaggtatcaccaaagaggacaaacgtccctgggatgaagcagcgtcctccacgcggattcaatccctatcatgcctacccttatagatcatacggggcaccgtatttcccaccgtacggatatgggagagttcccagattccgccggccgacacgctacaggccgtacttttga

SEQ ID NO: 6 - protein, 210 aa, Triticum aestivum
MDEEEHEVYGQEIPVDGDMEAADVDMTAAGDDAAKELDEMKRRLKEMEEEAAALRDMQAKVAKEMQGGDANASTAEAKEQVDARSVYVGNVDYACTPEEVQQHFQACGTVNRVTILTDKFGQPKGFAYVEFLEQEAVQEALNLNESELHGRQIKVSPKRTNVPGMKQRPPRGFNPYHAYPYRSYGAPYFPPYGYGRVPRFRRPTRYRPYF

SEQ ID NO: 7 - DNA, 20 bp, Artificial Sequence
  synthetic primer
aaaccttcccttctttcccg

SEQ ID NO: 8 - DNA, 20 bp, Artificial Sequence
  synthetic primer
tcagtacggtctgtagcgca

SEQ ID NO: 9 - DNA, 20 bp, Artificial Sequence
  synthetic primer
gattcggtttctagctcagc

SEQ ID NO: 10 - DNA, 20 bp, Artificial Sequence
  synthetic primer
gccacaacttagtagaaggg

SEQ ID NO: 11 - DNA, 20 bp, Artificial Sequence
  synthetic primer
ttagatcggagagagacggc

SEQ ID NO: 12 - DNA, 20 bp, Artificial Sequence
  synthetic primer
ttcatggaagcctcgtcctc

SEQ ID NO: 13 - DNA, 20 bp, Artificial Sequence
  synthetic primer
ataatcatcgcaagaccggc

SEQ ID NO: 14 - DNA, 20 bp, Artificial Sequence
  synthetic primer
gattcggtttctagctcagc

SEQ ID NO: 15 - DNA, 20 bp, Artificial Sequence
  synthetic primer
gccacaacttagtagaaggg

SEQ ID NO: 16 - DNA, 20 bp, Artificial Sequence
  synthetic primer
ttagatcggagagagacggc

SEQ ID NO: 17 - DNA, 20 bp, Artificial Sequence
  synthetic primer
ttcatggaagcctcgtcctc

(19) United States
(12) Patent Application Publication
(10) Pub. No.: US 2015/0211016 A1
(43) Pub. Date: Jul. 30, 2015

(54) ENVIRONMENTAL STRESS-RESISTANT PLANT WITH HIGH SEED PRODUCTIVITY AND METHOD FOR PRODUCING THE SAME

(71) Applicants: Ryozo IMAI, (US); Myung Hee KIM, (US); Yozo NAGIRA, (US); Naoki TAOKA, (US)

(72) Inventors: Ryozo Imai, Sapporo-shi (JP); Myung Hee Kim, Sapporo-shi (JP); Yozo Nagira, Takasago-shi (JP); Naoki Taoka, Takasago-shi (JP)

(73) Assignee: KANEKA CORPORATION, Osaka-shi, Osaka (JP)

(21) Appl. No.: 14/383,804

(22) PCT Filed: Mar. 8, 2013
(86) PCT No.: PCT/JP2013/057320

§ 371 Date: Sep. 8, 2014

(30) Foreign Application Priority Data
Mar. 8, 2012 (JP) 2012-052018

(51) Int. Cl. C12N 15/82; C07K 14/415

(52) U.S. Cl. CPC C12N 15/8273; C12N 15/8261; C07K 14/415

(57) ABSTRACT

This invention relates to a plant having high resistance to environmental stress and high seed productivity, and, more particularly, a transformed plant that is genetically modified to overexpress a polyadenylate-binding protein (PABN) gene and has enhanced environmental stress resistance and enhanced seed productivity.

### TECHNICAL FIELD

The present invention relates to a plant with high environmental stress resistance and high seed productivity and a method for producing the same.

### BACKGROUND ART

Environmental stress by drought or salt significantly influences plant growth. Also, freezing stress causes serious cell damage in plants. As a result, such environmental stresses significantly influence crop productivity. Therefore, various types of research and development aimed at impartation of environmental stress resistance to crops have been conducted.

In recent years, transgenic plants into which a variety of genes had been introduced have been produced via plant transformation technology. For example, it is reported that a plant into which an enzyme gene that synthesizes an amino acid proline has been introduced becomes tolerant to drought or salt stress because of the osmoregulatory function provided by proline (Non-Patent Document 1). It is also reported that through introduction of a transcription factor gene that regulates stress responses stress responses can be activated, and drought, salt, and low-temperature stress resistance can be imparted to a plant (Non-Patent Document 2). It is disclosed that through overexpression of genes encoding RNA-binding proteins, further, transgenic plants with low-temperature stress resistance can be produced (Patent Document 1).

Overexpression of transcription factor genes, however, results in overexpression of all genes induced by the transcription factor genes. As a result, even if the resulting plant acquires environmental stress resistance, adverse effects on growth may occasionally be observed (e.g., plant dwarfism) (Non-Patent Document 3). Therefore, discovery of novel genes that impart environmental stress resistance without causing adverse effects due to the overexpression thereof and improvement in techniques of transgene expression have been awaited.

Many people throughout the world eat grains as staples, and it is a critical object to improve the productivity thereof. While attempts have been made to impart environmental stress resistance to grain plants, development of grain plants having high productivity, in addition to environmental stress resistance, has been desired.

### CITATION LIST

Patent Documents
Patent Document 1: JP 2007-282542 A
Non-Patent Documents
Non-Patent Document 1: Kishor P, Hong Z, Miao G H, Hu C, and Verma D., Plant Physiol., August 1995; 108 (4): 1387-1394
Non-Patent Document 2: Liu Q, Kasuga M, Sakuma Y, Abe H, Miura S, Yamaguchi-Shinozaki K, and Shinozaki K., Plant Cell, August 1998, 10 (8): 1391-406; Jaglo-Ottosen K. R., Gilmour S. J., Zarka D. G., Schabenberger O., Thomashow M. F., Science, April 3, 1998; 280 (5360): 104-6
Non-Patent Document 3: Liu Q, Kasuga M, Sakuma Y, Abe H, Miura S, Yamaguchi-Shinozaki K, and Shinozaki K., Plant Cell, August 1998; 10 (8): 1391-406

### SUMMARY OF THE INVENTION

### Problem to Be Solved by the Invention

It is a problem underlying the present invention to provide a plant with high environmental stress resistance and high seed productivity.

Means for Solving the Problem

The present inventors have conducted concentrated studies in order to solve the above problem. As a result, they discovered that both environmental stress resistance and seed productivity could be effectively enhanced via introduction of the PABN gene into a plant. This has led to the completion of the present invention.

Specifically, the present invention includes the followings.

[1] A transformed plant having enhanced environmental stress resistance and enhanced seed productivity, which is genetically modified to overexpress a polyadenylate-binding protein (PABN) gene.

[2] The transformed plant according to [1], into which at least one of the following PABN genes (a) to (f) has been introduced:

(a) a gene comprising the nucleotide sequence as shown in SEQ ID NO: 1, 3, or 5;

(b) a gene comprising DNA hybridizing under stringent conditions to DNA consisting of the nucleotide sequence as shown in SEQ ID NO: 1, 3, or 5 and encoding a protein having polyadenylate-binding activity;

(c) a gene encoding a protein comprising the amino acid sequence as shown in SEQ ID NO: 2, 4, or 6;

(d) a gene encoding a protein comprising an amino acid sequence derived from the amino acid sequence as shown in SEQ ID NO: 2, 4, or 6 by deletion, substitution, or addition of one or several amino acids and having polyadenylate-binding activity;

(e) a gene comprising a nucleotide sequence having 85% or more identity to the nucleotide sequence as shown in SEQ ID NO: 1, 3, or 5 and encoding a protein having polyadenylate-binding activity; and

(f) a gene encoding a protein comprising an amino acid sequence having 90% or more identity to the amino acid sequence as shown in SEQ ID NO: 2, 4, or 6 and having polyadenylate-binding activity.

[3] The transformed plant according to [1] or [2], wherein the environmental stress resistance is salt stress resistance, drought stress resistance, and/or freezing stress resistance.

[4] The transformed plant according to any of [1] to [3], which is a grain plant or an oilseed plant.

[5] A method for enhancing both environmental stress resistance and seed productivity of a plant comprising introducing a polyadenylate-binding protein (PABN) gene into a plant cell and selecting a transformed plant in which environmental stress resistance and seed productivity have been enhanced.

[6] The method according to [5], wherein the PABN gene is at least one of the following (a) to (f):

(a) a gene comprising the nucleotide sequence as shown in SEQ ID NO: 1, 3, or 5;

(b) a gene comprising DNA hybridizing under stringent conditions to DNA consisting of the nucleotide sequence as shown in SEQ ID NO: 1, 3, or 5 and encoding a protein having polyadenylate-binding activity;

(c) a gene encoding a protein comprising the amino acid sequence as shown in SEQ ID NO: 2, 4, or 6;

(d) a gene encoding a protein comprising an amino acid sequence derived from the amino acid sequence as shown in SEQ ID NO: 2, 4, or 6 by deletion, substitution, or addition of one or several amino acids and having polyadenylate-binding activity;

(e) a gene comprising a nucleotide sequence having 85% or more identity to the nucleotide sequence as shown in SEQ ID NO: 1, 3, or 5 and encoding a protein having polyadenylate-binding activity; and

(f) a gene encoding a protein comprising an amino acid sequence having 90% or more identity to the amino acid sequence as shown in SEQ ID NO: 2, 4, or 6 and having polyadenylate-binding activity.

[7] The method according to [5] or [6], wherein the environmental stress resistance is salt stress resistance, drought stress resistance, and/or freezing stress resistance.

[8] The method according to any of [5] to [7], wherein the plant is a grain plant or an oilseed plant.

This description includes the content as disclosed in Japanese Patent Application No. 2012-052018 to which the present application claims priority.

### Effects of the Invention

The present invention can provide a plant with high environmental stress resistance and high seed productivity.

### BRIEF DESCRIPTION OF THE DRAWINGS

FIG. 1 shows photographs demonstrating the results of an experiment on salt stress resistance of an AtPABN-introduced transformed Arabidopsis thaliana (i.e., PABN-overexpressing plant) and a wild-type strain grown at high salt concentration. FIG. 1A shows the results for the wild-type strain and FIG. 1B shows those for the PABN-overexpressing plant.

FIG. 2 shows photographs demonstrating the results of an experiment on drought stress resistance of the AtPABN-introduced transformed Arabidopsis thaliana (i.e., PABN-overexpressing plant) and the wild-type strain grown under drought conditions. FIG. 2A shows the results for the wild-type strain and FIG. 2B shows those for the PABN-overexpressing plant.

FIG. 3 shows a photograph demonstrating the results of an experiment on freezing stress resistance of the AtPABN-introduced transformed Arabidopsis thaliana (i.e., PABN-overexpressing plant) and the wild-type strain grown under freezing conditions. FIG. 3A shows the results for the wild-type strain and FIG. 3B shows those for the PABN-overexpressing plant.

FIG. 4 shows a graph indicating the number of seeds per plant (seed productivity) obtained from the AtPABN-introduced transformed Arabidopsis thaliana (i.e., PABN-overexpressing plant) and the wild-type strain. The left bar shows the result for the wild-type strain and the right bar shows that for the PABN-overexpressing plant.

FIG. 5 shows a photograph showing the appearance of the AtPABN-introduced transformed Arabidopsis thaliana plant (i.e., PABN-overexpressing plant) and that of the wild-type strain.

FIG. 6 shows the structure of a vector for Agrobacterium transformation comprising the wheat PABN gene (the TaPABN1 or TaPABN2 gene).

FIG. 7 shows photographs indicating the results of expression analysis (RT-PCR) of a gene introduced into a TaPABN-overexpressing plant (Tᵢ generation).

FIG. 8 shows photographs indicating the results of an experiment on salt stress resistance of the TaPABN-overexpressing plant (T₁ generation).

### Embodiments for Carrying out the Invention

Hereafter, the present invention is described in detail.

### 1. The Transformed Plant of the Present Invention and Production Thereof

The present invention relates to a transformed plant that is genetically modified to overexpress a PABN gene. The transformed plant according to the present invention has enhanced environmental stress resistance and seed productivity.

The transformed plant according to the present invention may comprise the PABN gene introduced into the plant. Such transformed plant is also referred to as a transgenic plant.

### 1) PABN Gene and Production Thereof

A PABN gene encodes a polyadenylate-binding protein (PABN). In the present invention, the PABN gene may be Arabidopsis thaliana PABN gene (the AtPABN gene; also referred to as “AtPABN1”), or it may encode a gene encoding a PABN from another plant species and corresponding to the AtPABN gene. For example, the PABN gene may be from wheat. In the present invention, also, the PABN gene may be a variant of any of those genes. The PABN gene used in the present invention may be isolated from any of a variety of organism sources, including plants, animals, bacteria, and fungi. For example, a PABN gene preferably is from a grain plant or an oilseed plant.

An example of the PABN gene from Arabidopsis thaliana (i.e., the AtPABN gene) is the gene comprising the nucleotide sequence as shown in SEQ ID NO: 1. The nucleotide sequence as shown in SEQ ID NO: 1 encodes a protein comprising the amino acid sequence as shown in SEQ ID NO: 2.

An example of the PABN gene from wheat is the gene comprising the nucleotide sequence as shown in SEQ ID NO: 3 or 5. The nucleotide sequence as shown in SEQ ID NO: 3 encodes a protein comprising the amino acid sequence as shown in SEQ ID NO: 4 and the nucleotide sequence as shown in SEQ ID NO: 5 encodes a protein comprising the amino acid sequence as shown in SEQ ID NO: 6.

Further, the PABN gene according to the present invention may be a gene that comprises DNA hybridizing under stringent conditions to DNA consisting of the nucleotide sequence as shown in SEQ ID NO: 1, 3, or 5 and encoding a protein having polyadenylate-binding activity.

The PABN gene according to the present invention may be a gene that encodes a protein comprising the amino acid sequence as shown in SEQ ID NO: 2, 4, or 6. Alternatively, the gene may be a gene that encodes a protein comprising an amino acid sequence derived from the amino acid sequence as shown in SEQ ID NO: 2, 4, or 6 by deletion, substitution, or addition of one or several (2 to 9, and preferably 2 to 5) amino acids and having polyadenylate-binding activity.

Alternatively, the PABN gene according to the present invention may be a gene that comprises a nucleotide sequence having at least 85% or more, preferably 90% or more, more preferably 95% or more, and particularly preferably 98% or more (e.g., 99% or more) identity to the nucleotide sequence as shown in SEQ ID NO: 1, 3, or 5 and encodes a protein having polyadenylate-binding activity. Also, the PABN gene according to the present invention may be a gene that encodes a protein comprising an amino acid sequence having at least 90% or more, preferably 95% or more, more preferably 97% or more, and further preferably 98% or more (e.g., 99% or more) identity to the amino acid sequence as shown in SEQ ID NO: 2, 4, or 6 and having polyadenylate-binding activity.

According to the present invention, the term “stringent conditions” refers to conditions under which a specific nucleic acid hybrid is formed between two nucleic acids having at least 85% or more, preferably 90% or more, more preferably 95% or more, and particularly preferably 98% or more (e.g., 99% or more) sequence identity from each other but no hybrid is formed between nucleic acids having identity at a level lower than the levels mentioned above. More specifically, for example, under stringent conditions the reaction conditions include a sodium salt concentration of 15 to 750 mM, preferably 50 to 750 mM, and more preferably 300 to 750 mM; a temperature of 25° C. to 70° C., and more preferably 55° C. to 65° C.; and a formamide concentration is 0% to 50%, and more preferably 35% to 45%. Under stringent conditions, following hybridization, it is preferred that a filter be washed at a sodium salt concentration of 15 to 600 mM, preferably 50 to 600 mM, and more preferably 300 to 600 mM; and at temperature of 50° C. ably 60° C. to 65° C.

The term “gene” used herein can refer to DNA or RNA. Examples of DNA include genomic DNA and cDNA and RNA encompasses mRNA and the like. The PABN gene according to the present invention may comprise a sequence of an untranslated region (UTR) or a transcription regulatory region, in addition to a sequence of an open reading frame of the PABN gene.

Whether or not the PABN gene according to the present invention encodes a protein having polyadenylate-binding activity can be determined by expressing an expression vector comprising such gene incorporated thereinto in an adequate host and examining polyadenylate-binding activity of the expressed protein. The polyadenylate-binding activity of a protein can be examined in accordance with a conventional technique. A specific example is a technique for detecting binding of RI-labeled polyadenylate (Sachs, A. B., and R. D. Kornberg, 1985, Nuclear Polyadenylate-Binding Protein, Mol. Cell. Biol., 5: 1993-1996).

A person skilled in the art can obtain the PABN gene according to the present invention with the use of the nucleotide sequence as shown in SEQ ID NO: 1, 3, or 5 or a known sequence of the PABN gene, from genomic DNA, or RNA, including mRNA, extracted from a cell, in accordance with a conventional technique.

For example, cDNA synthesized by a conventional reverse transcription technique from mRNA extracted from a tissue or cell from an organism (e.g., a plant leaf) in accordance with a conventional technique is used as a template, PCR amplification is performed with primers designed on the basis of the nucleotide sequence of the known PABN gene. Thus, a DNA fragment comprising the PABN gene can be obtained.

A nucleotide sequence of the resulting DNA fragment comprising the PABN gene can be modified via, for example, site-directed mutagenesis. A mutation can be introduced into DNA by a known technique, such as the Kunkel method or the Gapped duplex method, or an equivalent technique thereto. Mutagenesis can be carried out with a site-directed mutagenesis kit, such as the Mutan®-Super Express Kit (TAKARA BIO INC.) or the LA PCR™ in vitro Mutagenesis Series Kit (TAKARA BIO INC.).

The DNA fragment comprising the PABN gene obtained as described above may be cloned into a vector in accordance with a conventional technique. When the PABN gene is introduced into a plant by the Agrobacterium method, the PABN gene is preferably cloned into an Agrobacterium-derived plasmid-based vector capable of introducing a target gene into a plant via Agrobacterium, for example, a binary vector. Examples of vectors that are preferably used include pBI, pPZP, and pSMA-based vectors. Use of pBI-based binary vectors or intermediate vectors is particularly preferred, and examples include pBI121, pBI101, pBI101.2, and pBI101.3 vectors. A binary vector is a shuttle vector that is capable of replicating in Escherichia coli and Agrobacterium. By infecting a plant with an Agrobacterium bacterium carrying the binary vector, a DNA region sandwiched between the LB sequence and the RB sequence of the vector (border sequences) (i.e., T-DNA) can be incorporated into a plant genome (EMBO Journal, 10 (3), 697-704, 1991). When a binary vector plasmid is used, the PABN gene may be inserted into a site between the LB sequence and the RB sequence of the binary vector. Alternatively, the PABN gene may be incorporated into a pUC-based vector, such as a pUC18, pUC19, or pUC9 vector, so as to directly introduce the target gene into a plant. Also, a plant virus vector, such as a Cauliflower mosaic virus (CaMV), Bean golden mosaic virus (BGMV), or Tobacco mosaic virus (TMV), may be used.

In order to insert the PABN gene into a vector, for example, purified DNA is first cleaved with adequate restriction enzymes, and inserted into a restriction enzyme site or multi-cloning site of an adequate vector DNA and ligated to the vector. It is necessary that the PABN gene be incorporated into a vector in a manner such that the gene is overexpressed in a target plant. To this end, the PABN gene is preferably incorporated downstream of a promoter or enhancer in the vector.

As the “promoter,” any promoter having a function of regulating expression of a gene downstream thereof in a plant cell can be used. For example, the promoter may induce expression in a manner specific for particular tissue or a particular developmental stage of a plant (i.e., a tissue-specific promoter or developmental stage-specific promoter), it may constantly induce expression in any plant tissue at any developmental stage (i.e., a constitutive promoter), or it may induce expression in the presence of a particular inducer (i.e., an inducible promoter). The promoter may or may not originate from a plant. Specific examples include Cauliflower mosaic virus (CaMV) 35S promoter, Nopaline synthase gene promoter (Pnos), Maize-derived ubiquitin promoter, rice-derived actin promoter, and tobacco-derived PR protein promoter. An example of the enhancer is an enhancer region that is used for improvement of expression efficiency of a target gene and comprises an upstream sequence in the CaMV35S promoter.

A vector also preferably comprises a terminator, a poly A addition signal, a 5′-UTR sequence, a marker gene, and the like, in addition to the PABN gene. As a terminator, a sequence that terminates the gene transcription induced by the promoter may be used. Examples thereof include a nopaline synthase (NOS) gene terminator, an octopine synthase (OCS) gene terminator, and a CaMV 35S RNA gene terminator. Examples of marker genes include a kanamycin resistance gene, a gentamicin resistance gene, a vancomycin resistance gene, a neomycin resistance gene, a hygromycin resistance gene, a puromycin resistance gene, a zeocin resistance gene, a blasticidin resistance gene, a dihydrofolate reductase gene, and an ampicillin resistance gene.

2) Production of Plant that is Genetically Modified to Overexpress the PABN Gene

In the present invention, a plant that is genetically modified to overexpress the PABN gene can be produced by introducing the PABN gene obtained above into a plant to produce a transformed plant. In the present invention, alternatively, a mutation that enhances expression of the endogenous PABN gene in a plant may be introduced into the genome of the plant. For example, a mutation that would induce a higher level expression may be introduced into a promoter of the endogenous PABN gene in a plant.

In the present invention, a plant in which the PABN gene is to be overexpressed may be a monocotyledonous or dicotyledonous plant. Examples of monocotyledonous plants include, but not limited to, plants that belong to Gramineae such as rice, barley, wheat, maize, sugarcane, zoysia, sorghum, Setaria italica, and Echinochloa esculenta; Liliaceae such as Asparagus officinalis, Lilium, Allium cepa, Allium tuberosum, and Etythronium japonicum; and Zingiberaceae such as Zingiber officinale, Zingiber mioga, and Curcuma longa. Examples of dicotyledonous plants include, but not limited to, plants that belong to Brassicaceae such as Arabidopsis thaliana, Brassica oleracea, rapeseed, cauliflower, broccoli, and Japanese radish; Solanaceae such as tomato, eggplant, potato, and tobacco; Leguminosae such as soybean, pea, bean, and alfalfa; Cucurbitaceae such as cucumber, melon, and pumpkin; Umbelliferae such as carrot, celery, and Cryptotaenia japonica; Compositae such as lettuce; Malvaceae such as cotton and okra; Chenopodiaceae such as sugar beet and spinach; Myrtaceae such as Eucalyptus and clove; and Salicaceae such as poplar. Since the transformed plant according to the present invention has improved seed productivity, in addition, use of a grain plant or an oilseed plant as a plant in which the PABN gene is to be overexpressed is also preferred in the present invention. In the present invention, the term “grain plant” refers to a plant that produces edible seeds, and such plant typically belongs to Gramineae. Examples of grain plants include wheat, barley, rye and the like, and rice and maize. The term “oilseed plant” refers to a plant that produces oilseeds, i.e., seeds having high oil and fat content and being used as starting materials for producing oil. Examples of oilseed plants include rapeseed, sesame, soybean, peanut, safflower, and cotton.

The PABN gene can be introduced into a plant by a general plant transformation technique, including Agrobacterium method, particle gun method, electroporation, polyethylene glycol (PEG) method, microinjection, and protoplast fusion method. These plant transformation techniques are described in detail in ordinary textbooks, such as “New Edition, Experimental Protocols for Model Plants, Genetic Techniques to Genomic Analysis” (under the supervision of Isao Shimamoto and Kiyotaka Okada, 2001 Shujunsha Co., Ltd.) and literature such as Hiei Y. et al., “Efficient transformation of rice (Oryza sativa L.) mediated by Agrobacterium and sequence analysis of the boundaries of the T-DNA.,” Plant J., 1994, 6, 271-282, and Hayashimoto, A. et al., “A polyethylene glycol-mediated protoplast transformation system for production of fertile transgenic rice plants.,” Plant Physiol., 1990, 93, 857-863.

In the case of using the Agrobacterium method, the PABN gene may be incorporated into a vector suitable for the Agrobacterium method, the resulting plant expression vector may be introduced into an adequate Agrobacterium strain (e.g., Agrobacterium tumefaciens) in accordance with a conventional technique (e.g., freeze-thawing), and the resulting bacterial strain may be inoculated into a plant for infection. Thus, the PABN gene can be incorporated into the plant cell genome. Examples of the Agrobacterium method include a variety of techniques, such as inoculation of Agrobacterium bacteria into a protoplast, inoculation thereof into a tissue or cell culture, or inoculation thereof directly into a plant (i.e., the in planta method). When a protoplast is used, a protoplast can be infected with Agrobacterium by a method involving a co-culture with Agrobacterium bacteria comprising a Ti plasmid or a method involving a fusion with a spheroplasted Agrobacterium bacterium (i.e., the spheroplast method). When a tissue or cell culture is used, a sterile cultured leaf section (leaf disc) or callus of the target plant may be infected with Agrobacterium. According to the in planta method, Agrobacterium may be directly inoculated into a seed or a part of plant (e.g., a bud) to infect the plant.

A plant infected with Agrobacterium is grown (and in the case of infection of a callus or the like, a plant is regenerated by a conventional technique) to produce seeds, the resulting seeds are collected, and the plants obtained therefrom are subjected to self-crossing two or more times. Then, by selecting a transformed plant carrying the PABN gene in homozygous form, a transformed plant that comprises the PABN gene introduced thereinto can be obtained.

Alternatively, when a gene is introduced into a plant by particle gun method, for example, a gene transfer apparatus (e.g., PDS-1000, BIO-RAD) is used in accordance with the manufacturer's instructions, to bombard metal particles coated with a DNA fragment comprising the PABN gene into a sample, thereby introducing the gene into a plant cell to obtain a transformed cell of interest. In general, the bombardment is preferably performed at a pressure between approximately 450 psi and 2,000 psi at a distance approximately between 4 cm and 12 cm from the target. The transformed cell into which the PABN gene had been introduced is cultured in a selection medium in accordance with a conventional plant tissue culture technique, and surviving cells are cultured in a redifferentiation medium (containing phytohormone, such as auxin, cytokinine, gibberellin, abscisic acid, ethylene, or brassinoride, at an adequate concentration). Thus, a transformed plant into which the PABN gene has been introduced can be regenerated.

It is preferred that the introduced PABN gene be incorporated into the plant genome of the transformed plant according to the present invention. Alternatively, the transformed plant may carry the PABN gene, e.g., as an expression vector comprising it.

It is also preferred to confirm that the introduced PABN gene is expressed under general conditions (e.g., at 25° C.) in the resulting transformed plant.

In the context of the present invention, the term “transformed plant” encompasses “T₀ generation,” which is a just redifferentiated plant obtained via transformation treatment by infection with Agrobacterium, and also encompasses “T₁ generation,” which is a progeny grown from seeds of the T₀ generation plant; “T₂ generation;” and further progeny plants thereof.

The transformed plant according to the present invention may comprise the PABN gene introduced into its genome in heterozygous form or preferably in a homozygous form. The transformed plant according to the present invention also encompasses a plant in which the PABN gene has been introduced into only some cells of the plant (i.e., a chimeric plant). However, it is more preferred that the transformed plant carries the introduced PABN gene in all plant cells.

The transformed plant of the present invention refers to any of whole plant, plant organs (such as a root, stem, leaf, petal, seed, or fruit), plant tissues (such as the epidermis, phloem, parenchyma, xylem, or vascular bundle), and cultured plant cells (such as a callus).

In the transformed plant into which the PABN gene has been introduced as described above, the PABN gene is overexpressed. The term “overexpression of the PABN gene” used herein means that the PABN gene expression is detected at a level that significantly exceeds the expression level of the endogenous PABN gene in a wild-type strain of the relevant plant. When the level of polyadenylate-binding activity measured in a protein extract from a biological sample of the transformed plant is significantly higher than that in a protein extract from a biological sample of a non-transformed plant, for example, the PABN gene is determined to be overexpressed.

A transformed plant that is genetically modified to overexpress the PABN gene obtained as described above (typically, a transformed plant into which the PABN gene has been introduced) has enhanced environmental stress resistance and enhanced seed productivity.

### 2. Evaluation of Environmental Stress Resistance and Seed Productivity

The transformed plant according to the present invention has enhanced environmental stress resistance. Specifically, the transformed plant according to the present invention preferably has at least one of salt stress resistance, drought stress resistance, and freezing stress resistance.

Salt stress resistance is an ability that enables a plant to grow with a higher viability even at high salt concentrations. Salt stress resistance can be evaluated by, for example, adding salt at a high concentration (for example, at a high salt concentration at which a non-transformed plant cannot survive or at which it would survive with a viability of less than 5%) to a medium, growing a plant for a given period of time, and then determining viability of the plant. For example, a plant may be grown in an MS medium supplemented with NaCl (200 mM) for a week and then its viability may be determined. The transformed plant according to the present invention can survive with a viability that is preferably 5% or higher, more preferably 10% or higher, further preferably 30% or higher, and still further preferably 50% or higher (e.g., 60% or higher) than that of a non-transformed plant under salt stress conditions, although the viability is not limited to the above levels.

Drought stress resistance is an ability that enables a plant to grow with a higher viability even under the circumstances of low moisture contents. Drought stress resistance can be evaluated by, for example, lowering the moisture content in a medium (for example, by lowering the moisture content to a level at which a non-transformed plant cannot survive or at which it would survive with a viability of less than 5%), growing a plant for a given period of time, and then determining the viability of the plant. The transformed plant according to the present invention can survive with a viability that is preferably 5% or higher, more preferably 10% or higher, further preferably 30% or higher, and still further preferably 50% or higher (e.g., 70% or higher) than that of a non-transformed plant under drought stress conditions, although the viability is not limited to the above levels.

Freezing stress resistance is an ability that enables a plant to grow with a higher viability even at a freezing temperature. Freezing stress resistance can be evaluated by, for example, lowering the cultivation temperature to below 0° C. (for example, by lowering the cultivation temperature to a temperature at which a non-transformed plant cannot survive or at which it would survive with a viability of less than 5%), growing a plant to grow therein for a given period of time, and determining the resulting viability. The transformed plant according to the present invention can survive with a viability that is preferably 5% or higher, more preferably 10% or higher, and further preferably 30% or higher than that of a non-transformed plant under freezing stress conditions, although the viability is not limited to the above levels.

These environmental stress resistances can be evaluated in accordance with, for example, the methods described in the Examples below. The environmental stress resistances result from PABN gene overexpression (for example, an increased expression level resulting from introduction of the PABN gene).

The transformed plant according to the present invention has enhanced seed productivity. Regardless of whether the environmental stress is applied or not thereto, the transformed plant according to the present invention is capable of producing seeds at a high yield. The transformed plant according to the present invention can produce seeds at a number of seeds increased by 10% or higher, preferably 20% or higher, more preferably 30% or higher, and further preferably 40% or higher than that of a non-transformed plant.

The seed productivity can be evaluated in accordance with, for example, the methods described in the Examples below. The high seed productivity of the transformed plant according to the present invention results from PABN gene overexpression (for example, an increased expression level resulting from introduction of the PABN gene).

Accordingly, the present invention relates to a method for producing a transformed plant having both enhanced environmental stress resistance and enhanced seed productivity as described above and a method for enhancing environmental stress resistance and seed productivity of a plant, characterized in that PABN gene is overexpressed (for example, by introduction of the PABN gene into a plant). More particularly, the present invention provides a method for enhancing both environmental stress resistance and seed productivity of a plant comprising, for example, introducing the PABN gene into a plant cell, and regenerating a plant if needed, and selecting a transformed plant in which environmental stress resistance and productivity have been enhanced.

According to the method of the present invention, both environmental stress resistance (such as salt stress resistance, drought stress resistance, and freezing stress resistance) and seed productivity can be enhanced by overexpression of the PABN gene in a plant without developing negative traits, such as dwarfism. Thus, the method of the present invention is very advantageous.

### EXAMPLES

Hereafter, the present invention is described in greater detail with reference to the following examples, although the technical scope of the present invention is not limited to these examples.

### Example 1

### Production of a PABN-introduced transformed Arabidopsis thaliana

1. cDNA Synthesis

Total RNA was extracted from a rosette leaf of Arabidopsis thaliana (Columbia-0) using the RNeasy Mini Kit (Qiagen). With the use of the obtained RNA, cDNA was synthesized using the PCR Core Kit (Applied Biosystems).

2. Isolation of AtPABN Gene Segment from cDNA

With the use of cDNA synthesized above as a template, PCR was carried out using the forward and reverse primers shown below.

Forward primer:
(SEQ ID NO: 7)
5′-AAACCTTCCCTTCTTTCCCG-3′
Reverse primer:
(SEQ ID NO: 8)
5′-TCAGTACGGTCTGTAGCGCA-3′

These forward and reverse primers were designed to amplify a region from 5′UTR to the termination codon of the known nucleotide sequence (GenBank Accession Number NM_{—}001203582) of the AtPABN gene (AtPABN1; At5G51120). PCR was carried out in 50 μl reaction system. A PCR reaction solution was prepared by mixing 0.2 μl of Ex Taq DNA polymerase (5 units/μl, TAKARA BIO INC.), 5 μl of 10× polymerase buffer (containing MgCl₂), 2.5 μl of 2.5 mM dNTPs solution, 0.1 μl of each primer (10 pmol/μl), and 2 μl of cDNA synthesized above (about 1 μg/μl), and the total amount of the reaction solution was adjusted to 50 μl with Milli-Q water. The PCR conditions and the number of the reaction cycles are summarized in Table 1 below.

TABLE 1

| | Reaction | Reaction | | Number of |
|---|---|---|---|---|
| Step of reaction | temperature | duration | | cycles |
| Denaturation | 94° C. | 4 | min | 1 |
| Denaturation | 94° C. | 30 | sec | 35 |
| Annealing | 55° C. | 30 | sec | |
| Extension | 72° C. | 1.5 | min | |
| Extension | 72° C. | 5 | min | 1 |

After the completion of PCR, the PCR product was examined by electrophoresis, and the amplification of a nucleic acid fragment of the predicted length (about 700 nucleotides) was observed. The obtained fragment was subjected to cloning using the pGEM-Teasy Vector Systems (Promega), and a plurality of positive clones were obtained. DNA inserts contained in these positive clones were sequenced using the BigDye Terminator v1.1 Cycle Sequencing Kit (Applied Biosystems) and the ABI DNA Sequencer (3130 DNA Sequencer). The determined nucleotide sequence was analyzed compared with the known sequence of the known AtPABN gene described above, thereby verifying that the cloned DNA insert was an AtPABN gene (Arabidopsis thaliana PABN gene). The obtained nucleotide sequence of the AtPABN gene ORF is shown in SEQ ID NO: 1 and the amino acid sequence of the protein encoded thereby is shown in SEQ ID NO: 2.

3. Introduction of AtPABN Gene into Arabidopsis thaliana using Agrobacterium

The AtPABN gene isolated as described above was inserted in the sense direction downstream of the CaMV35S promoter in the Ti-based vector pBI 121 vector (Clonetech).

At the outset, the plasmid prepared by inserting the AtPABN gene into the pGEM-Teasy vector as described above was treated with XhoI and SacI to prepare an XhoI-SacI fragment. The resulting XhoI-SacI fragment containing the AtPABN gene was ligated into a pBI 121 vector digested with XhoI and SacI in the sense direction. The resulting nucleic acid construct was transformed into Agrobacterium (GV3101/Pmp-90) via freeze-thawing (Hofgen et al., Storage of competent cells for Agrobacterium transformation, Nucleic Acids Res., Oct. 25, 1998; 16 (20): 9877). In order to select a transformed Agrobacterium strain, the Agrobacterium strains subjected to gene transfer were screened on a YEP medium containing kanamycin at 50 mg/l and gentamicin at 100 mg/l for kanamycin resistance and gentamicin resistance. The selected colonies were cultured in 5 ml of YEP medium containing kanamycin and gentamicin for 20 to 24 hours, inoculated into 100 ml of YEP medium containing kanamycin and gentamicin, and cultured until O.D.₆₀₀ reached 0.80. The bacterial culture solution was centrifuged at 5000× g for 10 minutes at room temperature. The precipitated bacterial cells were lysed in 50 ml of floral dropping medium. The lysate was injected into an Arabidopsis thaliana bud 3 to 5 times. The Agrobacterium-injected plant was put in a plastic bag and allowed to grow at 22° C. in the dark overnight. On the following day, the plant was transferred to long-day conditions at 22° C. Watering to the plant was started 3 days after the infection of the plant with the bacteria. The compositions of the media used in the experiment are as shown in Tables 2 and 3.

TABLE 2
YEP medium (pH 7.0)

| | |
|---|---|
| 10 g/L | Yeast extract |
| 10 g/L | Bactopeptone |
| 5 g/L | NaCl |

TABLE 3
Floral dropping medium

| | | |
|---|---|---|
| 2.3 | g/L | MS inorganic salt (Wako Pure Chemical Industries, Ltd.) |
| 1 | ml/L | 1000x MS vitamin* |
| 50 | g/L | Sucrose |
| 0.5 | ml/L | Transformation reagent Silwet L-77 |
| 200 | μl/L | 44 nM BAP |

*1000x MS vitamin (1 L): 100 g of myo-inositol, 2 g of L-glycin, 0.5 g of nicotinic acid, 0.5 g of pyridoxine HCl, and 0.5 g of thiamine HCl

### 4. Selection of AtPABN-Introduced Transformed Plant

Seeds were obtained from Arabidopsis thaliana,which had been transformed as described above and grown, and sterilized in a sterile solution (70% ethanol and 0.5% Triton X-100) for 30 minutes, and then further sterilized in 100% ethanol for 2 minutes. Thereafter, the seeds were sowed in MS medium containing kanamycin at 50 mg/l and vancomycin at 200 mg/l. T₂ generation plants resulting from self-pollination between plants that had been normally grown from the seeds in the medium (i.e., T₁ generation) were observed to show a segregation ratio of 3:1 between transformed plants and wild-type strains. Further, T₃ generation plants carrying the transgene in homozygous form were selected. The composition of the medium used in the experiment is as shown in Table 4.

TABLE 4
MS medium (pH 5.8)

| | | |
|---|---|---|
| 4.6 | g/L | MS inorganic salt (Wako Pure Chemical Industries, Ltd.) |
| 1 | ml/L | 1000x MS vitamin* |
| 20 | g/L | Sucrose |
| 8 | g/L | Agar |

*1000x MS vitamin (1 L): 100 g of myo-inositol, 2 g of L-glycin, 0.5 g of nicotinic acid, 0.5 g of pyridoxine HCl, and 0.5 g of thiamine HCl

In the selected AtPABN-introduced transformed plant, expression of the transgene was confirmed based on the expression of a drug-resistant gene, which indicated that the transformed plant had overexpressed the PABN gene.

### Example 2

### Evaluation of Environmental Stress Resistance

### 1. Experiment on Salt Stress Resistance

Seeds (25 each) of the AtPABN-introduced transformed Arabidopsis thaliana plants (PABN-overexpressing plants) prepared above and wild-type Arabidopsis thaliana strain (Columbia-0) were sowed in MS medium (2% sucrose, 8% agar; pH 5.7 to 5.8). The seeds were allowed to grow at 22° C. under continuous light conditions for one week, transferred to MS medium supplemented with NaCl (200 mM), and then allowed to grow at 22° C. under continuous light conditions for 4 days. Thereafter, the viability thereof was determined.

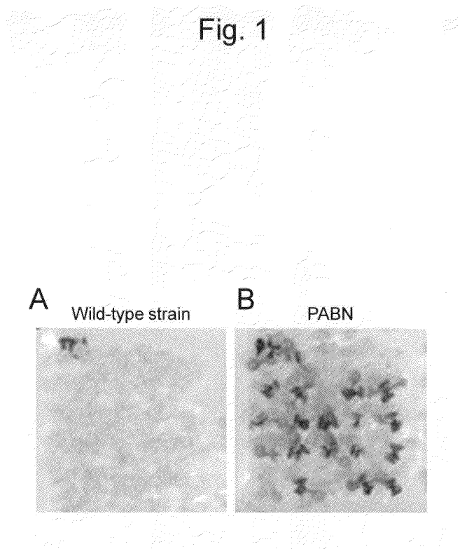

The results of the experiment on salt stress resistance are shown in FIG. 1. While no wild-type strain survived (viability: 0%), the AtPABN-introduced transformed Arabidopsis thaliana plants (“PABN” in the Figure) survived with a viability of 60%. This indicates that salt tolerance (salt stress resistance) was remarkably improved in a transformed plant overexpressing a PABN gene.

### 2. Experiment on Drought Stress Resistance

Seeds (48 each) of the AtPABN-introduced transformed Arabidopsis thaliana plants (PABN-overexpressing plants) prepared above and the wild-type Arabidopsis thaliana strain (Columbia-0) were sowed in MS medium (2% sucrose, 8% agar; pH 5.7 to 5.8). The seeds were allowed to grow at 22° C. under continuous light conditions for 10 days. Thereafter, the plants were transferred to soil (culture soil:vermiculite=3:1) and allowed to grow at 22° C. under short-day conditions (10 hours in the light and 14 hours in the dark). Water was not applied on the 3rd day and the 4th day after the initiation of cultivation on soil, and watering was started again on the 5th day. Viability was determined on the 10th day after the initiation of cultivation on soil.

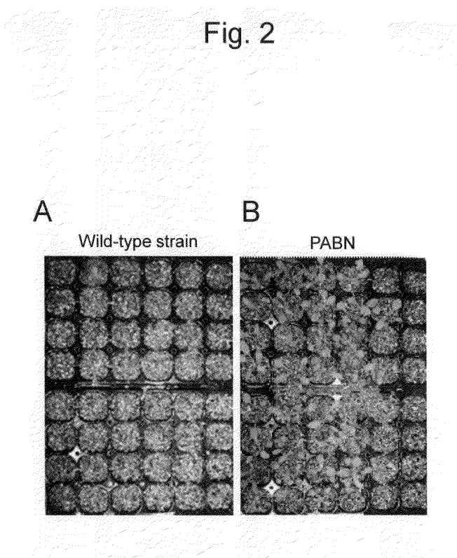

FIG. 2 shows the results of the experiment on drought stress resistance. While the wild-type strains showed viability of 2%, the PABN-overexpressing plants (“PABN” in the Figure) showed significantly higher viability (i.e., 79%) than the wild-type plants. This indicates that drought tolerance (drought stress resistance) was remarkably improved in the PABN-overexpressing transformed plants.

### 3. Experiment on Freezing Stress Resistance

Seeds (12 each) of the AtPABN-introduced transformed Arabidopsis thaliana plants (PABN-overexpressing plants) prepared above and the wild-type Arabidopsis thaliana strain (Columbia-0) were sowed in MS medium (2% sucrose, 8% agar; pH 5.7 to 5.8). The seeds were allowed to grow at 22° C. under continuous light conditions for 2 weeks, transferred to soil (culture soil:vermiculite =1:2), and then allowed to grow at 22° C. under short-day conditions (8 hours in the light and 16 hours in the dark) for one week. Thereafter, seeds were conditioned at low temperature of 4° C. for one week under short-day conditions. Subsequently, the transformed plants were placed in a programmed freezer and cultured at −2° C. for 1 hour. For the purpose of ice nucleation, the transformed plants were sprayed with tap water, and subsequently cooled with a program that the temperature was lowered by 1° C. every 2 hours to −14° C., and then cultured at 4° C. for 12 hours. Thereafter, the plants were cultured under short-day conditions at 22° C. for one week and the viability of the plants was determined.

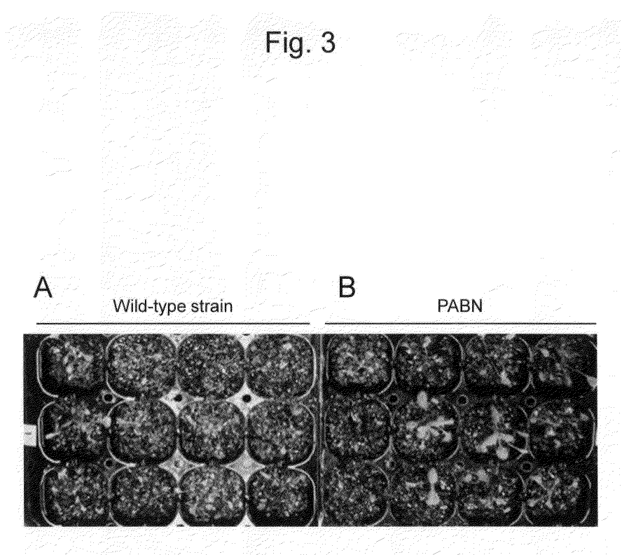

FIG. 3 shows the results for the experiment on freezing stress resistance. While no wild-type strains survived (viability: 0%), the PABN-overexpressing plants (“PABN” in the Figure) showed a significantly higher viability (i.e., 33.3%) than the wild-type plants. This indicates that freezing stress resistance was remarkably improved in the PABN-overexpressing transformed plants.

### Example 3

### Evaluation of Seed Productivity

The effect of the AtPABN gene transfection on seed productivity was tested. Seeds of the AtPABN-introduced transformed Arabidopsis thaliana (PABN-overexpressing plants) prepared above and the wild-type Arabidopsis thaliana strain (Columbia-0) were sowed in MS medium (2% sucrose, 8% agar; pH 5.7 to 5.8). The seeds were allowed to grow at 22° C. under long-day conditions (16 hours in the light and 8 hours in the dark) for about one month. These growth conditions were general conditions which include no addition of environmental stress, such as salt, drought, or freezing stress. After the seeds were grown, the seeds were collected, and the total number of seeds produced per plant was determined.

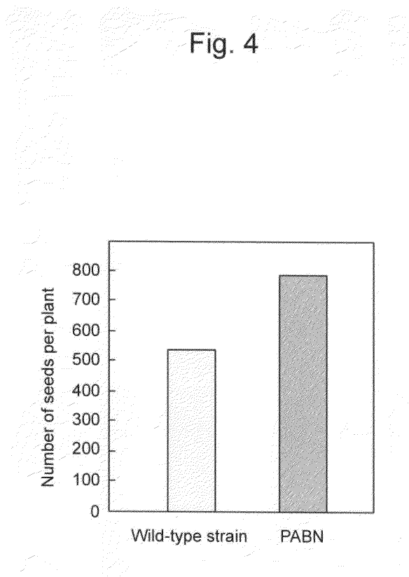

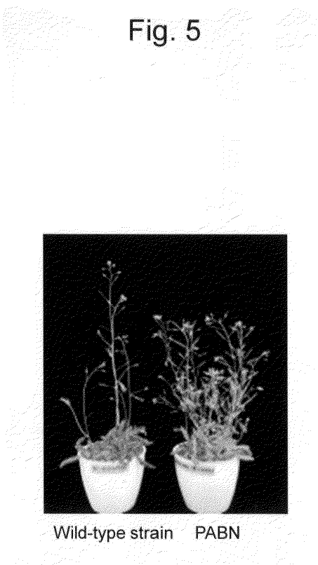

FIG. 4 shows the results. The number of seeds per plant was 500 seeds for the wild-type strain. In contrast, 788 seeds were obtained in the PABN-overexpressing plant (“PABN” in the Figure), which is 1.46 times greater than that of the wild-type strain. Scape branching was stimulated in the PABN-overexpressing plant and the number of siliques was increased (FIG. 5). This result indicates that the PABN gene stimulates seed formation to a significant degree and enhances seed productivity.

### Example 4

### Production of Transformed Wheat with Wheat PABN Gene

Transformed wheat plants in which two wheat orthologous genes of the AtPABN gene, TaPABN1 gene (GenBank Accession number: AK331378) and the TaPABN2 gene (GenBank Accession number: AK335747), had been overexpressed were produced in the manner described below.

1. cDNA Synthesis

Total RNA was extracted from a young leaf of a wheat (Triticum aestivum, cultivar: Yumechikara) using the RNeasy Mini Kit (Qiagen). With the use of the obtained RNA, cDNA was synthesized using the PCR Core Kit (Applied Biosystems).

2. Isolation of Wheat PABN Gene Segment from cDNA

With the use of cDNA synthesized above as a template, each gene was amplified via PCR using the forward and reverse primers shown below.

### Primer Set for TaPABN1 Amplification

TaPABN1 forward primer:
(SEQ ID NO: 9)
5′-GATTCGGTTTCTAGCTCAGC-3′
TaPABN1 reverse primer:
(SEQ ID NO: 10)
5′-GCCACAACTTAGTAGAAGGG-3′

### Primer Set for TaPABN2 Amplification

TaPABN2 forward primer:
(SEQ ID NO: 11)
5′-TTAGATCGGAGAGAGACGGC-3′
TaPABN2 reverse primer:
(SEQ ID NO: 12)
5′-TTCATGGAAGCCTCGTCCTC-3′

These sets of forward and reverse primers were each designed to amplify a region from 5′UTR to the termination codon of the two genes (TaPABN1 and TaPABN2). PCR was carried out in 50 μl reaction system. A PCR reaction solution was prepared by mixing 0.2 μl of Ex Taq DNA polymerase (5 units/μl, TAKARA BIO INC.), 5 μl of 10× polymerase buffer (containing MgCl₂), 2.5 μl of 2.5 mM dNTPs solution, 0.1 μl of each primer (10 pmol/μl), and 2 μl of cDNA synthesized above (about 1 μg/μl), and the total amount of the reaction solution was adjusted to 50 μl with Milli-Q water. The PCR conditions and the number of the reaction cycles shown in Table 1 in Example 1 were employed herein.

After the completion of PCR, the PCR product was examined by electrophoresis, and the amplification of a nucleic acid fragment of the predicted length (about 650 nucleotides) was observed. The obtained fragment was subjected to cloning using the pGEM-Teasy Vector Systems (Promega) and a plurality of positive clones were obtained. DNA inserts contained in these positive clones were sequenced using the BigDye Terminator v3.1 Cycle Sequencing Kit (Applied Biosystems) and the ABI DNA Sequencer (3130 DNA Sequencer). The determined nucleotide sequences were analyzed compared with the nucleotide sequences of TaPABN1 and TaPABN2 described above, thereby verifying that the cloned DNA inserts were TaPABN1 and TaPABN2. The obtained nucleotide sequence of the TaPABN1 ORF is shown in SEQ ID NO: 3 and the amino acid sequence of the protein encoded thereby is shown in SEQ ID NO: 4. Also, the obtained nucleotide sequence of the TaPABN2 gene ORF is shown in SEQ ID NO: 5 and the amino acid sequence of the protein encoded thereby is shown in SEQ ID NO: 6.

3. Introduction of Wheat PABN Gene into Wheat Using Agrobacterium

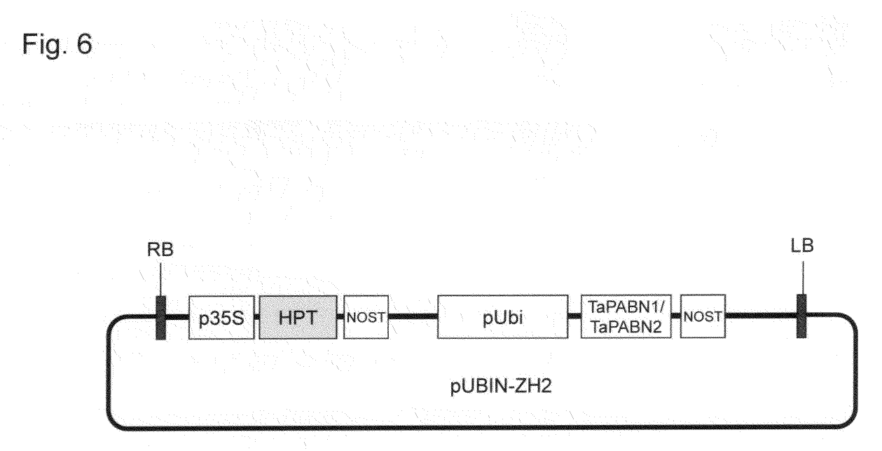

A fragment containing a wheat PABN gene (either of the TaPABN1 gene or the TaPABN2 gene above) was cleaved with restriction enzymes BamHI and KpnI from a plasmid prepared by inserting the wheat PABN gene into the pGEM-Teasy vector, and the resulting fragment was inserted into the enzyme cleavage site in the multicloning site of the binary vector pUBIN-ZH2. The binary vector pUBIN-ZH2 has a cassette comprising a hygromycin-resistant gene between the cauliflower mosaic virus 35S promoter and the nopaline synthase terminator and a gene expression cassette comprising a multicloning site between the maize ubiquitin gene promoter (Plant physiology, Volume 100, 1992, pp. 1503-1507) and the nopaline synthase terminator, within the T-DNA region of pPZP202 (P. Hajdukiewicz, Z. Svab, and P. Maliga, 1994, Plant Molecular Biology, 25: 989-994). Thus, two types of vectors for Agrobacterium transformation, each comprising a relevant wheat PABN gene, were produced (FIG. 6).

An Agrobacterium strain (LBA4404) was transformed with the obtained relevant transformation vector via freeze-thawing (Hofgen et al., Storage of competent cells for Agrobacterium transformation, Nucleic Acids Res., Oct. 25, 1998; 16 (20): 9877). Further, the transformation of wheat (cultivar: Yumechikara) was performed with either of the obtained Agrobacterium strain as described above. Wheat transformation was carried out via the in planta transformation technique as described in JP Patent Application No. 2005-513739.

### 4. Selection of Wheat PABN Transformed Plant

The genome was extracted from the T₁-generation plant obtained from the transformed wheat as described above using the Plant DNAzol reagent (Life Technologies). With the use of the extracted genome as a template, genomic PCR was carried out using the forward and reverse primers shown below. The reverse primer was designed on the nopaline synthase terminator.

### Primer Set for TaPABN1 Detection

TaPABN1 forward primer:
(SEQ ID NO: 9)
5′-GATTCGGTTTCTAGCTCAGC-3′
TaPABN1 reverse primer:
(SEQ ID NO: 13)
5′-ATAATCATCGCAAGACCGGC-3′

### Primer Set for TaPABN2 Detection

TaPABN2 forward primer:
(SEQ ID NO: 11)
5′-TTAGATCGGAGAGAGACGGC-3′
TaPABN2 reverse primer:
(SEQ ID NO: 13)
5′-ATAATCATCGCAAGACCGGC-3′

PCR was carried out in 50 μl reaction system. A PCR reaction solution was prepared by mixing 0.2 μl of Ex Tag DNA polymerase (5 units/μl, TAKARA BIO INC.), 5 μl of 10× polymerase buffer (containing MgCl₂), 2.5 μl of a 2.5 mM dNTPs solution, 0.1 μl of each primer (10 pmol/μl ), and 2 μl of cDNA synthesized above (about 1 μg/μl), and the total amount of the reaction solution was adjusted to 50 μl with Milli-Q water. The PCR conditions and the number of the reaction cycles shown in Table 1 in Example 1 were employed herein.

The transformed plants into which the wheat PABN gene had been introduced were selected by genomic PCR performed in the manner described above. In addition, the transformed plants were forced to self-pollinate, seeds were collected, and transformed wheat plants carrying the wheat PABN gene in homozygous form were selected.

### Example 5

### Confirmation of Expression of Transgene in T₁ Generation Plants of the Transformed Wheat

(1) Extraction of RNA from T₁ Transformant and Synthesis of cDNA

Leaves of the transformed plant of the T₁ generation (the first and second leaves) were collected in a microtube, and total RNA was extracted using the RNeasy Plant Mini Kit (QIAGEN) by the procedures in accordance with the instruction attached to the kit. With the use of 1 μg of the extracted total RNA, cDNA was synthesized via reverse transcription using the High Capacity RNA-to-cDNA™ Kit (Applied Biosystems).

### (2) Confirmation of Expression of Transgene in T₁ Generation Plants (via RT-PCR)

Transgene expression was confirmed via PCR using 1 μl of the cDNA solution prepared in Example 5(1). PCR was carried out under the conditions shown in Table 1, except that the number of cycles from the step of denaturation to the step of extension was changed to 30. PCR was carried out using the primers shown below.

TaPABN1 RT-PCR forward primer:
(SEQ ID NO: 14)
5′-GATTCGGTTTCTAGCTCAGC-3′
TaPABN1 RT-PCR reverse primer:
(SEQ ID NO: 15)
5′-GCCACAACTTAGTAGAAGGG-3′
TaPABN2 RT-PCR forward primer:
(SEQ ID NO: 16)
5′-TTAGATCGGAGAGAGACGGC-3′
TaPABN2 RT-PCR reverse primer:
(SEQ ID NO: 17)
5′-TTCATGGAAGCCTCGTCCTC-3′

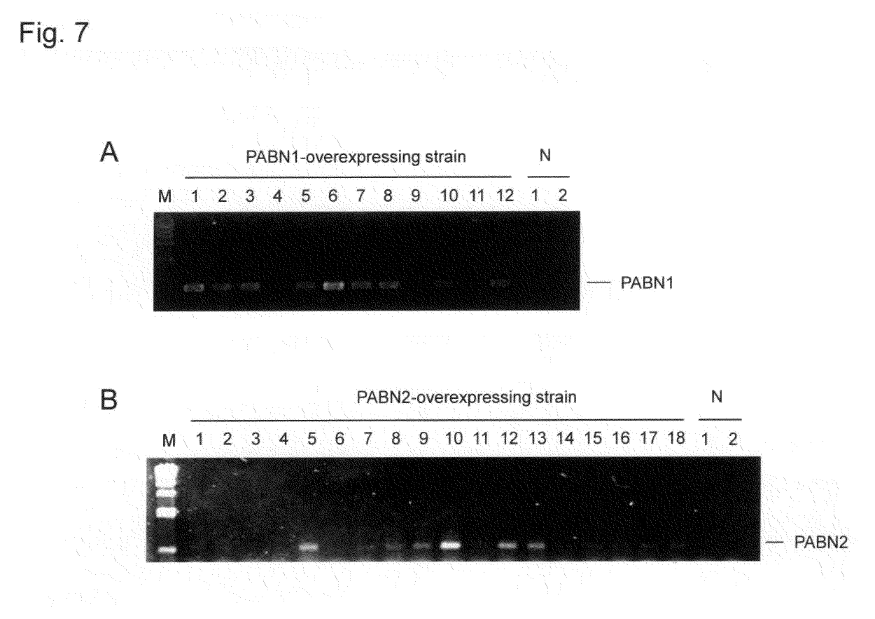

As shown in FIG. 7, plants expressing the transgene (i.e., TaPABN1- or TaPABN2-overexpressiong strains) were produced in the T₁ generation into which the TaPABN1 or TaPABN2 gene had been introduced. The fragment obtained by PCR was verified to be a TaPABN gene fragment via sequence analysis.

### Example 6

### Evaluation of Productivity of TaPABN Gene-Introduced Wheat

The effect of the introduction of the TaPABN gene on seed productivity was tested. Individual plants (seeds) of the TaPABN introduced wheat (the TaPABN2-overexpressing strains) prepared above and the wild-type wheat strain were sowed in culture soil and then allowed to grow at 22° C. under long-day conditions (16 hours in the light and 8 hours in the dark) for about one month. These growth conditions were general conditions which include no addition of environmental stress, such as salt, drought, or freezing stress. After the plants were grown, the number of branchings of each plant (i.e., the total number of branched stems) was determined. As the number of branchings increases, the number of spikes per plant increases and an increased yield can be expected.

Table 5 shows the results. The average number of branchings per individual plant was 3.4 in a wild-type strain (n=16), and it was 5.4 in the TaPABN2-overexpressing strains (n=8), which is 1.58 times greater than the number for the wild-type strain. This indicates that the TaPABN2 gene stimulates branching at a significant level and enhances seed productivity by 50% or more.

TABLE 5
Average number of branching per plant for TaPABN-
overexpressing transformed strains and wild-type strains
(values in parentheses represent the standard deviations)

| TaPABN2-overexpressing strain | Wild-type strain |
|---|---|
| 5.4 (±0.77) | 3.4 (±0.52) |

### Example 7

### Experiment on Salt Stress Resistance of TaPABN2-Introduced Wheat

Seeds (10 individuals each) of the TaPABN2-introduced wheat plants (the TaPABN2-overexpressing strains) and wild-type wheat strains were allowed to germinate in sterile water and then allowed to grow at 22° C. under long-day conditions for 3 days. Thereafter, the plants were allowed to grow in sterile water containing NaCl (400 mM) at 22° C. under long-day conditions for 2 days. The plants were transferred to culture soil, allowed to grow, and subjected to determination of viability 7 days later.

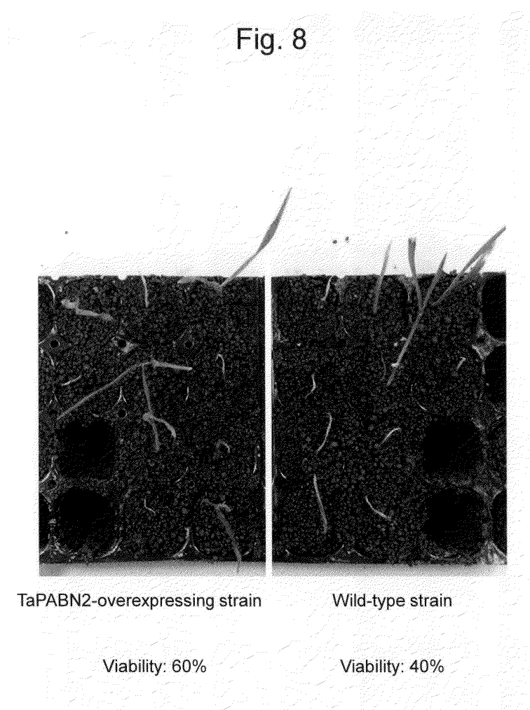

FIG. 8 shows photographs of the plants after the salt stress resistance experiment. While 4 wild-type strains survived (viability: 40%), 6 TaPABN2-overexpressing plants survived (viability: 60%). The results demonstrate that salt tolerance (salt stress resistance) is improved in transformed plants overexpressing the TaPABN2 gene.

### Example 8

### Production of AtPABN-Introduced Transformed Wheat

AtPABN-introduced transformed wheat plants were produced in the same manner as that described in Example 4, except that the Arabidopsis thaliana PABN gene obtained in Example 1 (i.e., the AtPABN gene) was used as a transgene.

The transformed plants into which the AtPABN gene had been introduced were selected with genomic PCR. In addition, the transformed plants was forced to self-pollinate, seeds were collected, and transformed wheat plants carrying the AtPABN gene in homozygous form were selected.

This transformed wheat has enhanced environmental stress resistance (salt stress resistance, drought stress resistance, and freezing stress resistance) and seed productivity, as does the PABN introduced transformed Arabidopsis thaliana plants produced in Example 1.

All publications, patents, and patent applications cited herein are incorporated herein by reference in their entirety.

### INDUSTRIAL APPLICABILITY

The present invention is applicable to production and cultivation of a plant having high environmental stress resistance and high seed productivity.

### SEQUENCE LISTING FREE TEXT

SEQ ID NOs: 7 to 17 show primers.

1. A transformed plant with enhanced environmental stress resistance and enhanced seed productivity, wherein said transformed plant is genetically modified to overexpress a polyadenylate-binding protein (PABN) gene.

2. The transformed plant according to claim 1, wherein at least one of the following PABN genes (a) to (f) has been introduced:
(a) a gene comprising the nucleotide sequence as shown in SEQ ID NO: 1, 3, or 5,
(b) a gene comprising DNA hybridizing under stringent conditions to DNA consisting of the nucleotide sequence as shown in SEQ ID NO: 1, 3, or 5 and encoding a protein having polyadenylate-binding activity,
(c) a gene encoding a protein comprising the amino acid sequence as shown in SEQ ID NO: 2, 4, or 6,
(d) a gene encoding a protein comprising an amino acid sequence derived from the amino acid sequence as shown in SEQ ID NO: 2, 4, or 6 by deletion, substitution, or addition of one or several amino acids and having polyadenylate-binding activity,
(e) a gene comprising a nucleotide sequence having 85% or more identity to the nucleotide sequence as shown in SEQ ID NO: 1, 3, or 5 and encoding a protein having polyadenylate-binding activity, and
(f) a gene encoding a protein comprising an amino acid sequence having 90% or more identity to the amino acid sequence as shown in SEQ ID NO: 2, 4, or 6 and having polyadenylate-binding activity.

3. The transformed plant according to claim 1, wherein the environmental stress resistance is salt stress resistance, drought stress resistance, and/or freezing stress resistance.

4. The transformed plant according to claim 1, wherein said transformed plant is a grain plant or an oilseed plant.

5. A method for enhancing both environmental stress resistance and seed productivity of a plant, said method comprising
introducing a polyadenylate-binding protein (PABN) gene into a plant cell, and
selecting a transformed plant in which environmental stress resistance and seed productivity are enhanced.

6. The method according to claim 5, wherein the PABN gene is at least one of the following (a) to (f):
(a) a gene comprising the nucleotide sequence as shown in SEQ ID NO: 1, 3, or 5,
(b) a gene comprising DNA hybridizing under stringent conditions to DNA consisting of the nucleotide sequence as shown in SEQ ID NO: 1, 3, or 5 and encoding a protein having polyadenylate-binding activity,
(c) a gene encoding a protein comprising the amino acid sequence as shown in SEQ ID NO: 2, 4, or 6,
(d) a gene encoding a protein comprising an amino acid sequence derived from the amino acid sequence as shown in SEQ ID NO: 2, 4, or 6 by deletion, substitution, or addition of one or several amino acids and having polyadenylate-binding activity,
(e) a gene comprising a nucleotide sequence having 85% or more identity to the nucleotide sequence as shown in SEQ ID NO: 1, 3, or 5 and encoding a protein having polyadenylate-binding activity, and
(f) a gene encoding a protein comprising an amino acid sequence having 90% or more identity to the amino acid sequence as shown in SEQ ID NO: 2, 4, or 6 and having polyadenylate-binding activity.

7. The method according to claim 5, wherein the environmental stress resistance is salt stress resistance, drought stress resistance, and/or freezing stress resistance.

8. The method according to claim 5, wherein the plant is a grain plant or an oilseed plant.

9. The method according to claim 5, wherein the plant is selected from rice, barley, wheat, rye, maize, sugarcane, zoysia, sorghum, Setaria italica, Echinochloa esculenta; Asparagus officinalis, Lilium, Allium cepa, Allium tuberosum, Etythronium japonicum; Zingiber officinale, Zingiber mioga, Curcuma longa, Arabidopsis thaliana, Brassica oleracea, rapeseed, sesame, soybean, peanut, safflower, cauliflower, broccoli, Japanese radish, tomato, eggplant, potato, tobacco, soybean, pea, bean, alfalfa, cucumber, melon, pumpkin, carrot, celery, Cryptotaenia japonica, lettuce, cotton and okra, sugar beet, spinach, Eucalyptus, clove, and poplar.

10. A method for producing a transformed plant having enhanced environmental stress resistance and enhanced seed productivity comprising
introducing a polyadenylate-binding protein (PABN) gene into a plant cell, and
selecting a transformed plant in which the environmental stress resistance and seed productivity has been enhanced.

11. The method according to claim 10, wherein the PABN gene is at least one of the following (a) to (f):
(a) a gene comprising the nucleotide sequence as shown in SEQ ID NO: 1, 3, or 5,
(b) a gene comprising DNA hybridizing under stringent conditions to DNA consisting of the nucleotide sequence as shown in SEQ ID NO: 1, 3, or 5 and encoding a protein having polyadenylate-binding activity,
(c) a gene encoding a protein comprising the amino acid sequence as shown in SEQ ID NO: 2, 4, or 6,
(d) a gene encoding a protein comprising an amino acid sequence derived from the amino acid sequence as shown in SEQ ID NO: 2, 4, or 6 by deletion, substitution, or addition of one or several amino acids and having polyadenylate-binding activity,
(e) a gene comprising a nucleotide sequence having 85% or more identity to the nucleotide sequence as shown in SEQ ID NO: 1, 3, or 5 and encoding a protein having polyadenylate-binding activity, and
(f) a gene encoding a protein comprising an amino acid sequence having 90% or more identity to the amino acid sequence as shown in SEQ ID NO: 2, 4, or 6 and having polyadenylate-binding activity.

12. The method according to claim 10, wherein the environmental stress resistance is salt stress resistance, drought stress resistance, and/or freezing stress resistance.

13. The method according to claim 10, wherein the plant is a grain plant or an oilseed plant.
